(19) United States
(12) Patent Application Publication
(10) Pub. No.: US 2021/0065904 A1
(43) Pub. Date: Mar. 4, 2021

(54) PERFORMING MEDICAL TASKS BASED ON INCOMPLETE OR FAULTY DATA

(71) Applicant: Siemens Healthcare GmbH, Erlangen (DE)

(72) Inventors: Thomas BOETTGER, Erlangen (DE); Ulrich HARTUNG, Langensendelbach (DE); Benedikt KRUEGER, Ebensfeld (DE); Dominik NEUMANN, Erlangen (DE); Maximilian WUERSTLE, Baiersdorf (DE)

(73) Assignee: Siemens Healthcare GmbH, Erlangen (DE)

(21) Appl. No.: 17/002,987

(22) Filed: Aug. 26, 2020

(30) Foreign Application Priority Data
Aug. 29, 2019 (DE) 10 2019 213 000.3

(51) Int. Cl. G16H 50/20; G16H 50/70; G16H 10/60; G16H 40/20; G06F 16/215; G06N 7/00; G06K 9/46; G06F 40/40

(52) U.S. Cl. CPC G16H 50/20; G16H 50/70; G16H 10/60; G16H 40/20; G06K 2209/01; G06N 7/005; G06K 9/46; G06F 40/40; G06F 16/215

(57) ABSTRACT

A computer-implemented method and a system are for performing or supporting a medical task. An embodiment of the method includes obtaining a medical task and obtaining values for data fields of a number of available data fields. The method further includes determining whether an insufficient data field is present; and, if such a field is present, determining a relevance metric for the medical task, for the insufficient data field and/or the value thereof. Further, the method includes providing, via an estimator function, at least two different values for the insufficient data field; calculating at least two results for the medical task, which are based on the at least two different values provided; determining whether the relevance metric determined reaches or exceeds a relevance threshold value and, if this is the case, outputting an output signal based on the at least two results calculated.

### PRIORITY STATEMENT

The present application hereby claims priority under 35 U.S.C. § 119 to German patent application number DE 102019213000.3 filed Aug. 29, 2019, the entire contents of which are hereby incorporated herein by reference.

### FIELD

Various example embodiments of the invention generally relate to a method for performing medical tasks based upon incomplete or faulty data.

### BACKGROUND

Medical tasks (or medical issues), for example diagnostic tasks, are increasingly performed or at least supported by software systems. These software systems rely heavily on data provided on individual patients (for example in electronic health records, EHR), on patient cohorts or even on the general population.

In many situations, not all the facts underlying a decision in a medical task or as a medical task can be accessed by a physician making the decision or by a software system that supports a physician's decision.

This can, for example, be due to the following reasons:

data has either not been acquired or has only been acquired to an insufficient extent (for example due to an error or because the patient was uncooperative or unconscious)

data is located on an inaccessible system (for example since the physician or software do not have the necessary access authorization for the system)

data is available, but is not plausible (data input errors, errors with optical character recognition (OCR), errors with natural language processing (NLP), translation errors, interpretation errors, obsolete data, etc.).

This can interrupt and/or delay the decision-making process, for example because data has to be (re-)acquired or read out manually from archives or other data storage media. For example, it may be necessary for the physician to make a telephone call to obtain the missing value or even to arrange an additional or repeat examination of a patient.

Even then, existing software systems often do not permit manual data input and this can result in additional complications or delays. Furthermore, in the event of at least one data field being empty (corresponding, for example, to a specific variable), known software systems will fail to continue to execute the intended task. This can result in the undesirable situation of physicians themselves having to make decisions without any support from the software system.

The U.S. Pat. No. 7,650,321 B2 from the prior art describes methods for handling missing data in medical decision support systems. This patent describes the use of a “global value” instead of the missing value or the use of the most probable value for a missing value. Furthermore, the patent describes selection methods for determining the most probable value.

### SUMMARY

However, the inventors have discovered that the methods in this patent do not give any indication of the inherent uncertainty in a medical task resulting from the artificial selection of the value of the missing parameter.

Embodiments of the present invention are directed to a computer-implemented method for performing and supporting a medical task and a computer system for performing or supporting a medical task with improved handling of missing or insufficient (or: faulty) input values.

This the subject matter of embodiments is set forth in the independent claims.

At least one embodiment of the present invention is directed to a computer-implemented method for performing or supporting a medical task is provided, the computer-implemented method comprising:

obtaining a medical task to be performed;

obtaining a plurality of values for a plurality of data fields of a number of available data fields related to medical data (for example patient data, medical history data, study data, data relating to permissible methods and/or acceptable guide values and the like);

determining (in particular automatically) whether, after the obtaining of the plurality of values, at least one insufficient data field is present, wherein an insufficient data field is a data field for which no value was obtained or for which the value obtained is insufficient according to at least one quality criterion, and, if at least one insufficient data field is present:

determining a relevance metric for the medical task, for at least one of the at least one insufficient data field and/or the value thereof;

providing (in particular calculating), by means of an estimator function, at least two different values for the at least one of the at least one insufficient data field;

calculating at least two results for the medical task to be performed, which are based on the at least two different values provided;

determining whether the relevance metric determined reaches or exceeds a relevance threshold value; and

outputting, upon the relevance metric reaching or exceeding a relevance threshold value, an output signal based on the at least two results calculated.

According to a further embodiment, the invention also provides a method for training an estimator function f_{θ} to be used in the method according to the first embodiment of the present invention. As training samples, values for specific data fields in originally complete sets of values for all data fields can be artificially distorted or omitted and the values which were originally present and are now missing from the actual respective training sample can then be used as a label for this sample.

Moreover, according to a second embodiment of the present invention, a computer system for performing or supporting a medical task is provided, comprising:

an output interface;

an input interface, which is embodied:
to obtain a medical task to be performed;
to obtain a plurality of values for a plurality of data fields from a number of available data fields, which are related to medical data;

a computing apparatus, which is embodied:
to determine whether, after the obtaining of the plurality of values, at least one insufficient data field is present, wherein an insufficient data field is a data field for which no value was obtained or for which a value was obtained which is insufficient according to at least one quality criterion, and, (at least) if at least one insufficient data field is present:
to determine a relevance metric of the at least one of the at least one insufficient data field and/or the value thereof for the medical task;
to use an estimator function to provide at least two different values provided for the at least one of the at least one insufficient data field;
to calculate at least two results for the medical task to be performed based on the at least two different values for the at least one of the at least one insufficient data field;
to determine whether the specific relevance metric is greater than or equal to a relevance threshold value; and
to control the output interface to output, upon the specific relevance metric being greater than or equal to the relevance threshold value, an output signal based on the at least two results calculated.

According to a third embodiment of the present invention, a computer program product is provided, which contains program code, which, when executed (for example by a computer system) executes the method according to the first embodiment of the present invention.

According to a fourth embodiment of the present invention, a non-volatile computer-readable data storage medium is provided, which contains program code, which is embodied, when executed (for example by a computer system), to execute the method according to the first embodiment of the present invention. The data storage medium can be a DVD, a CD-ROM, a solid-state drive (SSD), a memory stick and or the like.

According to a fifth embodiment of the present invention, a data stream is provided, which includes program code, or is embodied to generate program code, which, when executed (for example by a computer system), executes the method according to the first embodiment of the present invention.

At least one embodiment is directed to a computer-implemented method for performing or supporting a medical task, comprising:

obtaining a medical task to be performed;

obtaining a plurality of values for a plurality of data fields of a number of available data fields related to medical data;

determining whether, after the obtaining of the plurality of values for the plurality of data fields, at least one insufficient data field is present among the plurality of data fields, wherein an insufficient data field is a data field for which no value was obtained or for which a value obtained is insufficient according to at least one quality criterion;

determining, upon determining that at least one insufficient data field is present among the plurality of data fields, a relevance metric for the medical task to be performed, for at least one of the at least one insufficient data field and a value of the at least one insufficient data field;

providing, via an estimator function, at least two different values for the at least one of the at least one insufficient data field;

calculating at least two results for the medical task to be performed, the at least two results being based on the at least two different values provided;

determining whether the relevance metric determined reaches or exceeds a relevance threshold value; and

outputting, upon the determining indicating that the relevance metric reaches or exceeds the relevance threshold value, an output signal based on the at least two results calculated.

At least one embodiment is directed to a computer system for performing or supporting a medical task, comprising:

an output interface;

an input interface configured:
to obtain a medical task to be performed; and
to obtain a plurality of values from a plurality of data fields of a number of available data fields, related to medical data;

a computing apparatus configured:
to determine, after the obtaining of the plurality of values of the plurality of data fields, at least one insufficient data field is present among the plurality of data fields, wherein an insufficient data field is a data field for which no value was obtained or for which a value was obtained which is insufficient according to at least one quality criterion,
to determine, upon determining that at least one insufficient data field is present among the plurality of data fields, a relevance metric for at least one of the at least one insufficient data field and the value of the at least one insufficient data field, for the medical task;
to use an estimator function to provide at least two different values for the at least one of the at least one insufficient data field;
to calculate at least two results for the medical task to be performed based upon the at least two different values provided for the at least one of the at least one insufficient data field;
to determine whether the relevance metric determined is greater than or equal to a relevance threshold value, and
to control, upon the determining indicating that the relevance metric reaches or exceeds the relevance threshold value, the output interface to output an output signal based on the at least two results calculated.

At least one embodiment is directed to a non-transitory computer program product storing executable program code, which when executed by at least one processor, configures the at least one processor to perform the method of of an embodiment.

At least one embodiment is directed to a non-volatile, computer-readable data storage medium storing executable program code, which when executed by at least one processor, configures the at least one processor to perform the method of an embodiment.

### BRIEF DESCRIPTION OF THE DRAWINGS

The invention will be explained further in greater detail with reference to example embodiments, which are depicted in the attached drawings.

The attached drawings are appended in order to enable a better understanding of the embodiments of the present invention and represent part of the present disclosure. The drawings illustrate embodiments of the present invention and are intended, together with the description, to describe the principle of the invention in more detail. Other embodiments of the present invention and many of the intended advantages of the present invention will become apparent when they are described in more detail with reference to the drawings. Here, the same references designate the same or similar parts.

The numbering of method steps is intended to facilitate understanding and, unless explicitly stated otherwise or implicitly obvious, should not be interpreted as meaning that the designated steps have to be performed in accordance with the numbering of their references. In particular, some or even all of the method steps can be performed simultaneously, in an overlapping manner or successively.

FIG. 1 shows a schematic flow diagram for illustrating a computer-implemented method according to the first embodiment of the present invention;

FIG. 2 shows a schematic flow diagram for illustrating a computer system according to the second embodiment of the present invention;

FIG. 3 is a schematic illustration of possible interim results and final results of the method according to FIG. 1;

FIG. 4 shows a schematic block diagram for illustrating a computer program product according to the third embodiment of the present invention; and

FIG. 5 shows a schematic block diagram for illustrating a data storage medium according to the fourth embodiment of the present invention.

Although specific embodiments are illustrated and described herein, it should be understood that any of the embodiments described and or parts thereof can be interchanged without departing from the subject matter of the present invention. In particular, this description is intended to cover any modifications or variants of the specific example embodiments described herein.

### DETAILED DESCRIPTION OF THE EXAMPLE EMBODIMENTS

The above and other elements, features, steps, and concepts of the present disclosure will be more apparent from the following detailed description in accordance with example embodiments of the invention, which will be explained with reference to the accompanying drawings.

Some examples of the present disclosure generally provide for a plurality of circuits, data storages, connections, or electrical devices such as e.g. processors. All references to these entities, or other electrical devices, or the functionality provided by each, are not intended to be limited to encompassing only what is illustrated and described herein. While particular labels may be assigned to the various circuits or other electrical devices disclosed, such labels are not intended to limit the scope of operation for the circuits and the other electrical devices. Such circuits and other electrical devices may be combined with each other and/or separated in any manner based on the particular type of electrical implementation that is desired. It is recognized that any circuit or other electrical device disclosed herein may include any number of microcontrollers, a graphics processor unit (GPU), integrated circuits, memory devices (e.g., FLASH, random access memory (RAM), read only memory (ROM), electrically programmable read only memory (EPROM), electrically erasable programmable read only memory (EEPROM), or other suitable variants thereof), and software which co-act with one another to perform operation(s) disclosed herein. In addition, any one or more of the electrical devices may be configured to execute a program code that is embodied in a non-transitory computer readable medium programmed to perform any number of the functions as disclosed.

It is to be understood that the following description of embodiments is not to be taken in a limiting sense. The scope of the invention is not intended to be limited by the embodiments described hereinafter or by the drawings, which are taken to be illustrative only.

The drawings are to be regarded as being schematic representations, and elements illustrated in the drawings are not necessarily shown to scale. Rather, the various elements are represented such that their function and general purpose become apparent to a person skilled in the art. Any connection, or communication, or coupling between functional blocks, devices, components, or other physical or functional units shown in the drawings or described herein may also be implemented by an indirect connection or coupling. A communication between devices may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

At least one embodiment of the present invention is directed to a computer-implemented method for performing or supporting a medical task is provided, the computer-implemented method comprising:

obtaining a medical task to be performed;

obtaining a plurality of values for a plurality of data fields of a number of available data fields related to medical data (for example patient data, medical history data, study data, data relating to permissible methods and/or acceptable guide values and the like);

determining (in particular automatically) whether, after the obtaining of the plurality of values, at least one insufficient data field is present, wherein an insufficient data field is a data field for which no value was obtained or for which the value obtained is insufficient according to at least one quality criterion, and, if at least one insufficient data field is present:

determining a relevance metric for the medical task, for at least one of the at least one insufficient data field and/or the value thereof;

providing (in particular calculating), by means of an estimator function, at least two different values for the at least one of the at least one insufficient data field;

calculating at least two results for the medical task to be performed, which are based on the at least two different values provided;

determining whether the relevance metric determined reaches or exceeds a relevance threshold value; and

outputting, upon the relevance metric reaching or exceeding a relevance threshold value, an output signal based on the at least two results calculated.

If no insufficient data fields are present, the medical task can be performed in the usual manner based on all the values provided for the data fields.

An idea behind the present invention is that only certain missing or insufficient values estimated to be relevant lead to additional steps being performed (for example inquiries or information output), while others do not alter the method for supporting the physician or for performing the medical task at all or only alter it to a small extent. This means that a physician is not unnecessarily confronted with warning messages or pop-up questions if the insufficient data field (or its missing or insufficient value) do not ultimately significantly influence the task to be performed (or do not influence it in a relevant way).

For example, it may normally be necessary to complete a data field that describes a patient's age, a patient's weight or a patient's blood group, and if no such value is obtained (for example received) for this data field, this may result in the computer system requesting the physician to contribute this value. However, if, according to the present invention, it is established that, for a special task to be performed, for example to determine the risk of the patient developing a certain disease, the patient's age, weight or blood group is completely irrelevant (or sufficiently irrelevant to the relevance threshold value), then, for example, advantageously, no request may be generated for the physician, since this would only impede the workflow without producing any sufficient advantage.

Moreover, advantageously two results are calculated for the medical task to be performed using values provided by an estimator function f_{θ}, wherein θ is at least one optional parameter. For example, two values provided by the estimator function f_{θ} can be fed into a task function g representing the medical task to be performed and a result for the medical task (i.e. an output of the task function g) calculated for each of these values.

This enables the two results to be compared in order to determine whether and/or to what degree (i.e. how much) the at least two different values influence the medical task. In this way, a physician can be supplied with additional insights into the results and the degree of uncertainty thereof and the physician can obtain information as to how necessary it is to improve the insufficient data field.

The estimator function f_{θ} can be any type of learned function, for example a function derived from a machine learning method. For example, the estimator function f_{θ} can be based on linear or logistic regression, machine learning, support vector machines (SVM) and/or the like. The estimator function f_{θ} can be trained on the total population or on a subcohort. The estimator function f_{θ} can be trained to output one single value (instead of the at least two values) or to represent a plurality of values as described in the foregoing. A subcohort for a specific patient describes a set of people with one or more characterizing features (for example age, gender, pre-existing conditions) in common with the specific patient. The characterizing features can be specifically related to a specific medical task.

According to a further embodiment, the invention also provides a method for training an estimator function f_{θ} to be used in the method according to the first embodiment of the present invention. As training samples, values for specific data fields in originally complete sets of values for all data fields can be artificially distorted or omitted and the values which were originally present and are now missing from the actual respective training sample can then be used as a label for this sample.

The method steps do not have to be performed in the sequence in which they are named and can be performed in numerous variants in a different sequence and/or partially or completely simultaneously or in an overlapping manner.

The step of calculating the at least two results can be performed as part of the determination of the relevance metric. For example, if a—hypothetically or actually—relatively lower and relatively higher value (for example a minimum value and a maximum value) for the insufficient data field leads to results that deviate from one another by a percentage greater than a relevance threshold value, which is, for example, formed by a specific amount of percentage points (for example greater than 10%, greater than 20%, greater than 30% or the like), this can result in the insufficient data field being determined as relevant in the method. Then, for example, a warning signal (as a type of output signal) displaying the aforesaid difference to a physician in percentage points may be output. Hence, the physician can decide how the results should be interpreted, which measures should be taken and the like.

Even if the specific relevance metric does not exceed the relevance threshold value, an output signal may be output, wherein this output signal preferably has other properties. Despite this, the performance of (or support for) the medical task can be continued. However, in such cases, one single value for the insufficient data field may be provided automatically and one single result for the task function g used for the automatic calculation. This can be performed as, for example described in U.S. Pat. No. 7,650,321 B2, the entire contents of which are hereby incorporated herein by reference. The output signal can control a display or another type of output device to depict or present in some other way the result for the task function g, i.e. the response to the medical task obtained, as a result of which the medical task is or can be performed or supported.

The single value for the task function g can be based on a single value, which is provided by the estimator function f_{θ} or by another estimator function. Such an estimator function f_{θ} for providing a single value can be embodied to provide a constant value which was derived from population statistics. If the population is designated P, the result of the estimator function f_{θ} can be calculated as f_{θ}=avg(p: p∈P), i.e. as the average value for the insufficient data field, wherein the averaging includes all the people p in the population P, or it can be calculated as the median for the population f_{θ}=median(p: p∈P) and/or the like. For example, if the value obtained for the body mass index, BMI, is insufficient, the average body mass index, BMI or the median of the body mass index, BMI for the total population can be used as the result of the estimator function f_{θ}.

The estimator function f_{θ} for providing the single value can also be embodied to provide a constant value derived from a subcohort of the population P, which is characterized by information in the non-insufficient data fields. For example, here the estimator function f_{θ} used could be the average body mass index, BMI, or the median of the body mass index, BMI, of a subcohort having the same gender, comparable age, same smoking status and comparable height to those of the patient.

The single value can also be calculated to determine whether a specific value obtained for a data field is sufficient or insufficient. For example, a value can be classed insufficient if it deviates from the aforesaid single value by a difference greater than a threshold value (which can in turn be set as an absolute value threshold value or as a relative value threshold value).

For example, if a value for the data field “body mass index, BMI” of 350 is obtained, this value (or the data field that obtains this value) is assessed as insufficient (or to be more specific: as implausible) since, for example, the median of the subcohort for the patient for this data field is 37 and the difference between 350 and 27 is greater than a relative value threshold value of, for example, 20% difference or since the difference is greater than an absolute value threshold value of, for example, 10. An individual absolute value threshold value and/or relative value threshold value can be provided for each data field.

This can be particularly helpful in the case of data fields, which are known to be unreliable due to their data source types, for example optical character recognition, OCR, or natural language processing, NLP, or due to the fact that that these are usually entered manually in the patient's file (in other words: for data fields where spelling mistakes or conversion errors are more common than errors resulting from a lack of certainty, for example measurement uncertainties).

A medical (in particular diagnostic) task can, for example, be the determination of whether the patient has a specific disease or what the probability of this is. A medical prediction task can, for example, be the determination of the amount of time that will elapse until a specific medical event will take place with a predetermined probability, for example, the patient will change from the first stage of a disease to a second stage of the disease, until the patient develops a specific symptom, until the patient is cured or the like. In particular, a task can be: “How high is the risk of the patient suffering from coronary heart disease within the next five years?” or: “What risk does [a specific therapeutic or diagnostic procedure] entail for the patient?”.

Such tasks are usually dependent upon at least one variable (corresponding to a data field), which describes a state or a property of a patient, for example the patient's weight, height, or a body mass index, BMI. Values for such variables can be entered into corresponding data fields or be obtained for the corresponding data fields.

Values that are insufficient according to the at least one quality criterion can, for example, be values, which are classed as unreliable or implausible, for example after a plausibility analysis or because the data sources for them are included in a list of unreliable data sources.

A plausibility analysis can include comparing the value obtained for a specific patient with a mean value and/or the like for the total population and/or the patient's subcohort and to determine whether the value obtained is an outlier, which indicates that the value is implausible.

Unreliable data sources can, for example, be data sources which include the conversion of information from one type of medium or carrier signal into another, for example natural language processing, NLP (conversion of information from audio into written text) or optical character recognition, OCR (conversion of analog text into digital text) or from unstructured text into structured text and the like.

If the medical task is represented by a task function g (x, y), wherein x and y are one or more data fields (variables), wherein x designates sufficient data fields and y insufficient data fields, the result of g (x, y) can be the desired result for the medical task to be performed.

Since, however, y is insufficient in this example (i.e. has missing values or implausible or unreliable values), the actual result may be incalculable or unusable. Additional data fields (variables) x′ can be present, which are not directly relevant for the medical task and which are represented by the task function g. According to one of the fundamental ideas of the present description, the estimator function f_{θ} may be able to approximate and estimate y adequately by yₑₛₜ=f_{θ} (x, x′). Hence, the medical task can be performed by calculating g(x, f_{θ}(x, x′)).

The output signal can include or consist of a warning signal, for example a visual, acoustic and/or haptic warning signal. The warning signal can warn or inform a physician of the presence of an insufficient data field, of the relevance of the insufficient data field and/or the value thereof, of the difference (in absolute amounts and/or in percentage points) between the results based on the at least two values for the insufficient data field and/or the like.

Additionally or alternatively, the output signal can include or consist of a control signal, which initiates an automatic process for acquiring and/or improving the value for the at least one previously insufficient data field. For example, the control signal can initiate a workflow in a clinical project management system, which performs an examination on a patient, allows specific data to be entered manually, allows the patient to be called and asked specific questions, requests data from other data sources (for example from another entity such as, for example, another hospital or a research institute) and/or the like.

The control signal can also automatically control the entire workflow. The control signal can also pause a computer system, which performs the method for performing or supporting the medical task, (for example, in order to force the physician to provide their own diagnosis instead of using or considering a diagnosis, which was compiled by the computer system based on at least one relevant and insufficient data field).

The output signal or warning signal can in particular be embodied to display at least two calculated results for the medical task to a user/physician using a display device. The warning signal can display the uncertainty in the result for the medical task to be performed due to the at least one insufficient data field, for example in that a central value and at least one corresponding error bar are displayed (so that for example two or three results are shown).

In some preferred embodiments, variants or developments of embodiments, the calculation of the at least two results for the medical task to be performed includes the calculation of a result for each of the at least two different values provided for the at least one of the at least one insufficient data field.

In some advantageous embodiments, variants or developments of embodiments, the at least two different values are a minimum value yₘᵢₙ and a maximum value yₘₐₓ for the insufficient data field. This permits an in-depth estimate of the degree to which the insufficient data field and/or the actual value thereof (which can be unknown, either because the value was not obtained at all or because the value was obtained in an insufficient state or insufficient manner) influence the medical task. Even if it is determined that the data field or the value is relevant, a physician can still decide that overall the influence is small enough to enable the method or computer system to continue to be used to perform or at least support the medical task to be performed.

In some preferred embodiments, variants or developments of embodiments, the at least one insufficient data field is a data field having binary values or having values, which have a linear influence on the medical task to be performed (i.e. with which the task function g is linearly dependent upon the at least one insufficient data field). It is particularly simple to calculate the relevance (or influence) of such values on the medical task to be performed, so that the specific relevance metric and the results for the medical task based on the different values for the insufficient data field are particularly accurate.

Particularly in these cases, ceteris paribus, the minimum and the maximum value gₘᵢₙ and gₘₐₓ of the task function g are easy to calculate as a function of the at least one of the at least one insufficient data field in that the minimum value and the maximum value yₘᵢₙ, yₘₐₓ are used for the at least one insufficient data field.

In some advantageous embodiments, variants or developments of embodiments, the at least two different values provided by the estimator function f_{θ} are different quantiles. For example, the two different values can be different percentiles. Percentiles are special quantiles that split a distribution into 100 equal parts. Hence, for example, “0.5 quantile”, “50% percentile” and “median” designate the same size.

The two different values can, for example, be selected as at least one percentile over 50% (preferably greater than or equal to 75%, more preferably greater than or equal to 85%, still more preferably greater than or equal to 95%, even still more preferably greater than or equal to 99%) and at least one percentile smaller than 50% (preferably smaller than or equal to 25%, more preferably smaller than or equal to 15%, still more preferably smaller than or equal to 5%, even still more preferably smaller than or equal to 1%). The calculation and depiction of quantiles or percentiles (instead of, for example, minimal values and maximal values) has the advantage that outliers (which can, for example, be present due to significant errors within the datasets) have less effect on the results of the estimator function than is the case, for example, with an average value calculation.

In some advantageous embodiments, variants or developments of embodiments, the at least two results for the medical task to be performed and/or the at least two different values provided by the estimator function are based on a general population or on a subcohort including a patient for whom the medical task is to be performed. This can make the results more accurate. The patient's subcohort can advantageously be determined automatically based on the non-insufficient data fields.

In some advantageous embodiments, variants or developments of embodiments, the calculation of the at least two results for the medical task to be performed is performed based on a probability distribution for the at least one of the at least one insufficient data field. The probability distribution can be based on population statistics and/or a subcohort of a patient toward whom the medical task is directed. This allows an even more realistic estimation of the relevance of the insufficient data field for the result of the medical task to be performed.

In some advantageous embodiments, variants or developments of embodiments, if at least one of the at least two results calculated results or a size derived therefrom meets a predetermined condition, a warning signal and/or a control signal is automatically output, which indicates that it is necessary for an improved value for the at least one insufficient data field to be obtained (for example recovered, retrieved, input etc.) and/or which performs a control function so that an improved value of this kind is obtained. As already mentioned in the foregoing, output signals of this kind can perform a control function for messages to be played or depicted to a user (for example a physician) so that workflows are initiated, a database is automatically accessed, an examination is terminated and/or the like.

In some embodiments, such or similar signals can be sent in cases when it is established that the specific relevance metric is lower than the relevance threshold value (or greater than or equal to the relevance threshold value in other variants). In this way, if it is established that at least one data field is insufficient, measures can be taken to rectify this, even if the aforesaid insufficient data field is not relevant for the present medical task to be resolved.

In some advantageous embodiments, variants or developments of embodiments, the at least two different values for the at least one of the at least one insufficient data field result from a corresponding main value and the corresponding error bar thereof. Here, in the case of a value of 5±3 for example, the value of 5 is designated the main value or central value. In the aforesaid way, it is simple to estimate the range or spread of a specific value obtained for a data field using the intrinsic information on the accuracy of the value, which is encoded by the error bar or plurality of error bars. In some variants, at least three (or exactly three) different values can be provided, which include or consist of a given value (main value) and the extremes displayed by the error bar thereof. For example, if 11±2 is given as a value, it is possible for either minimum values 9 and 13 based on this to be used or for the values 9, 11 and 13 to be used.

In some advantageous embodiments, variants or developments of embodiments, one of the at least one quality criterion consists in whether a value was generated by optical character recognition, OCR, or natural language processing, NLP. In this way, specific data sources that are known to occasionally generate small, but difficult to identify, errors (for example a missing decimal point in the case of OCR) can be monitored more closely as a matter of principle.

This criterion, and also any other optional criteria that are part of the quality criterion, can be linked with any other criteria by logical connectors (AND, OR etc.). Each criterion can be a necessary criterion and/or a sufficient criterion.

In some advantageous embodiments, variants or developments of embodiments, the output signal indicates at least one part of a source (less preferably, the entire source) which was used for the OCR or the NLP and which has to be checked in order to improve the value or values obtained for the at least one insufficient data field. For example, in the case of OCR, a user, for example a physician, can be shown a sentence or a portion of a page containing the respective text which was processed by the OCR in order to generate the value for the insufficient data field (for example a value that was evaluate as missing, implausible or insufficient in another way) so that the user or physician can determine the correct value manually based on the text. In a similar way, in the case of NLP, an audio clip can be played (or prepared to be played on the instigation of the user) which includes natural language that was processed by the NLP in order to generate the value for the insufficient data field.

In some advantageous embodiments, variants or developments of embodiments, one of the at least one quality criterion consists in whether reliability information of optical character recognition, OCR, and/or natural language processing, NLP, as the source of a value is above a prespecified threshold value. Accordingly, for example, a value (according to a sufficient condition) can be classed as insufficient if it originates from OCR or NLP and (logic: AND) if additionally the reliability information for the value is below (or equal to) a prespecified threshold value. NLP and OCR algorithms are partially configured to output reliability information (or: confidence) themselves which indicate how reliable (or true to the original) the conversion effected is estimated to be. For example, an NLP algorithm can output that a specific NLP result is classed “as 95% correct”. Alternatively, the algorithm can also itself be given a reliability evaluation (as a type of reliability information), for example “this algorithm is on average 95% correct”.

The prespecified threshold value is preferably above 50%, more preferably above 75%, even more preferably above 90%, particularly preferably above 95% or even higher. It is also possible for individual threshold values to be set in dependence on a respective data field. For example, a higher threshold value could be set for a data field, which usually only contains one single word and/or which obtains words from a set of easily confused words than for words from a set of words that are easily distinguishable.

Moreover, according to a second embodiment of the present invention, a computer system for performing or supporting a medical task is provided, comprising:

an output interface;

an input interface, which is embodied:
to obtain a medical task to be performed;
to obtain a plurality of values for a plurality of data fields from a number of available data fields, which are related to medical data;

a computing apparatus, which is embodied:
to determine whether, after the obtaining of the plurality of values, at least one insufficient data field is present, wherein an insufficient data field is a data field for which no value was obtained or for which a value was obtained which is insufficient according to at least one quality criterion, and, (at least) if at least one insufficient data field is present:
to determine a relevance metric of the at least one of the at least one insufficient data field and/or the value thereof for the medical task;
to use an estimator function to provide at least two different values provided for the at least one of the at least one insufficient data field;
to calculate at least two results for the medical task to be performed based on the at least two different values for the at least one of the at least one insufficient data field;
to determine whether the specific relevance metric is greater than or equal to a relevance threshold value; and
to control the output interface to output, upon the specific relevance metric being greater than or equal to the relevance threshold value, an output signal based on the at least two results calculated.

The computer system, in particular the computing apparatus, can be configured to perform the medical task in the usual way based on the values provided for the data fields if no insufficient data field is present.

The input interface and/or the output interface can be embodied as hardware, for example as a circuit or as a printed circuit board, as a field-programmable gate array, FPGA and/or as an application-specific integrated circuit, ASIC, and/or using transistors, logic gates or other circuits. In addition, the input interface and/or the output interface can also at least partially be implemented as software. The input interface and/or the output interface can be embodied to obtain data via cables or wirelessly and to obtain it via any known communication protocol. In particular, the input interface and/or the output interface can be configured to communicate with a plurality of data sources, for example with a local user interface, a remote data storage location and/or a cloud computing system.

For example, the medical task to be performed can be input into the system via a local user interface of the input interface, together with information characterizing, a specific patient or a specific subcohort and, using the output interface, the system can request relevant data on the patient or the subcohort from a remote data storage location from which the input interface then obtains the aforesaid data for the plurality of data fields.

According to a third embodiment of the present invention, a computer program product is provided, which contains program code, which, when executed (for example by a computer system) executes the method according to the first embodiment of the present invention.

According to a fourth embodiment of the present invention, a non-volatile computer-readable data storage medium is provided, which contains program code, which is embodied, when executed (for example by a computer system), to execute the method according to the first embodiment of the present invention. The data storage medium can be a DVD, a CD-ROM, a solid-state drive (SSD), a memory stick and or the like.

According to a fifth embodiment of the present invention, a data stream is provided, which includes program code, or is embodied to generate program code, which, when executed (for example by a computer system), executes the method according to the first embodiment of the present invention.

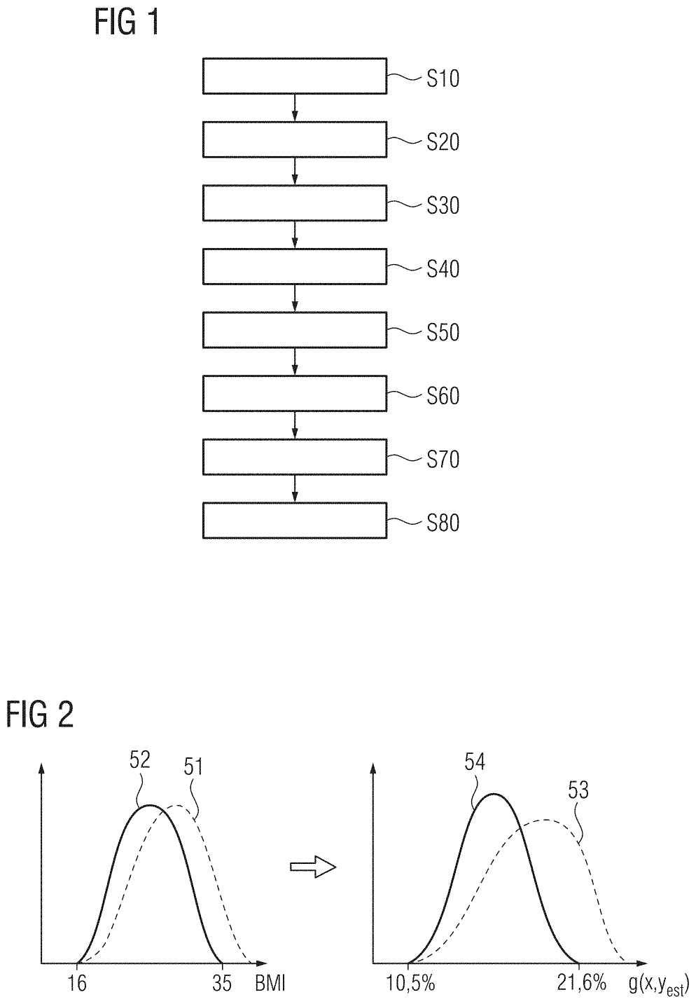

FIG. 1 shows a schematic flow diagram for illustrating a computer-implemented method according to the first embodiment of the present invention, i.e. a computer-implemented method for performing or supporting a medical task.

In the following, the method according to FIG. 1 is also partially described with reference to FIG. 2. FIG. 2 shows a schematic block diagram for illustrating a computer system 100 according to the second embodiment of the present invention, i.e. a computer system 100 for performing or supporting a medical task. The computer system 100 includes an input interface 110, an output interface 190 for outputting an output signal 71 and a computing apparatus 150.

References to the computer system 100 during the description of the method according to FIG. 1 are solely for purposes of illustration. Although the method according to FIG. 1 and each of its variants or developments can advantageously be performed with the computer system 100, it should be understood that the method according to FIG. 1 can also specifically performed without the computer system 100.

As an example, the following discusses a case in which, in addition to other data fields (i.e. variables), a medical task to be performed for a specific patient, represented by a task function g, is dependent on the patient's body mass index, BMI. The body mass index, BMI, is calculated by dividing the patient's weight (mass) in kilograms by the square of their height in meters. It should be understood that a plurality of other types of medical tasks can be performed and/or supported by the present method and that, as already explained in the foregoing, a plurality of variants, modifications and developments can be applied to the method.

In a step S10, a medical task to be performed is obtained (or initiated or provided), for example received by a computer system 100 via an input interface 110 of the computer system 100. The medical task can be input into a local user interface, for example by selecting a medical task to be performed and a patient for whom the medical task is to be performed at a physician's terminal. As an example used herein, the medical task can, for example, be worded: “How high is the risk of the patient suffering from coronary heart disease in the next five years?”.

In a step S20, a plurality of values for a plurality of data fields are obtained (or provided) from a number of available data fields, which are related to medical data. The available data fields can be any data fields, which are usually acquired and stored in electronic health records (EHR), for example gender, age, blood group, pre-existing diseases etc. The values can be read out automatically from a database, which can be arranged on the same site (i.e. the same location in the same entity as the local terminal) or which can be arranged remotely, for example a data memory of a research institution, a cloud computing system and/or the like.

In a step S30, it is determined whether at least one insufficient data field is present, i.e. whether any data field is still empty (here: missing body mass index, BMI) and whether every value obtained meets the at least one quality criterion.

As already described in the foregoing, the quality criterion can be a request for a specific plausibility evaluation after an automatic plausibility analysis of the values in the data fields and/or a request for a specific reliability evaluation, which was appended to the value when it was obtained, and/or a request for a specific type of data source for the value and/or the like.

The quality criterion can also be a threshold value for the size of the error bar associated with an obtained value. For example, at the error bar-threshold value, the main value (central value) can be used with good results so that the task function g can be calculated using the main value (central value). On the other hand, if the error bar is too large, it may be insufficient only to use the main value (central value). Instead of this, the corresponding data field may then be evaluated as insufficient.

As already mentioned, the following describes a case in which only a value for the data field “body mass index, BMI” is missing. The aforementioned medical task would typically take account of a patient's body mass index, BMI. If this value is missing, a decision support system from the prior art is not able to give a physician an answer for the aforesaid medical task based on the values obtained.

However, in the present embodiment, in a step S40, an estimator function f_{θ} is used to provide (for example calculate) at least two different values for the at least one of the at least one insufficient data field. If the insufficient data field is designated y, the at least two different values can be designated y₁, y₂, etc. The other data fields (which here are assumed not to be insufficient) are designated x. Hence, if the task function is designated g, g(x, y) must be determined to perform the medical task.

The estimator function f_{θ} can, for example, provide two values y₁, y₂, which correspond to a minimum value and a maximum value for y. In the present example, y₁ would be the minimum value for the body mass index, BMI, (either for the total population or for a subcohort to which the patient belongs) and y₂ the maximum value for the body mass index, BMI.

In a similar way, it is also possible to use quantiles (for example percentiles) instead, wherein preferably at least one percentile is over 50% (preferably greater than or equal to 75%, more preferably greater than or equal to 85%, still more preferably greater than or equal to 95%, even still more preferably greater than or equal to 99%) and one percentile is preferably smaller than 50% (preferably smaller than or equal to 25%, more preferably smaller than or equal to 15%, still more preferably smaller than or equal to 5%, even still more preferably smaller than or equal to 1%).

For example, in the step S40, the estimator function f_{θ} could determine that possible values for the body mass index, BMI, based on a subcohort for the patient are between 16 and 35.

Following this, in a step S50, a result for the task function g, g₁=g(x, y₁) and g₂=g(x, y₂) is calculated for each of the at least two values y₁, y₂ of the estimator function f_{θ}. In particular, if g is a non-linear function in y, a probability distribution or at least one property of the probability distribution of g (for example the mean value, a standard deviation or specific quantiles) can be calculated using Monte Carlo simulations.

In other variants, the estimator function f_{θ} can output a probability distribution yₑₛₜ for the value for the insufficient data field, preferably based on population statistics and/or a subcohort (or subcohort statistics). Hence, in the present example a probability distribution for the body mass index, BMI, can be provided.

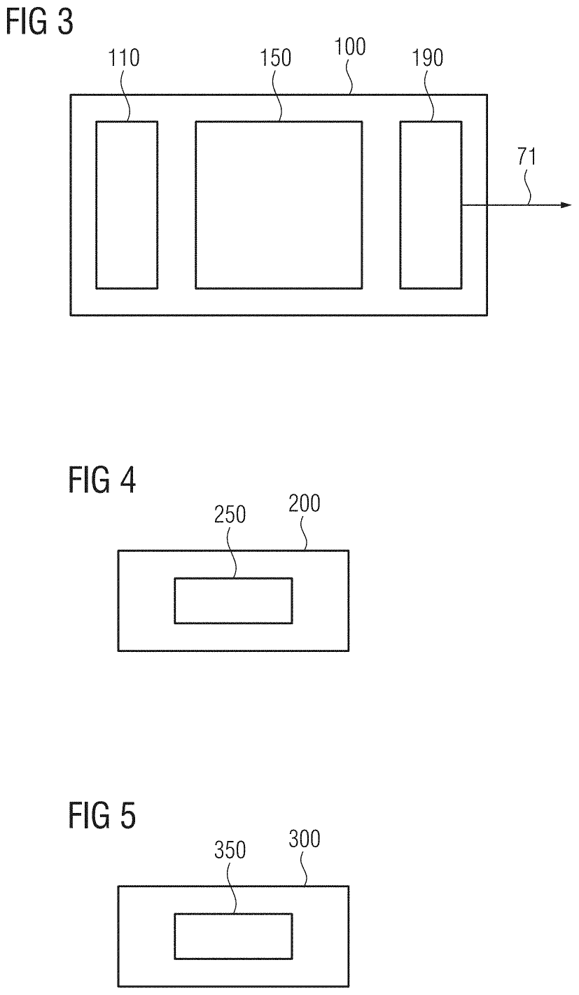

FIG. 3 is a schematic illustration of this variant. The left-hand side of FIG. 3 shows a probability distribution 51 for the body mass index, BMI, the general population and a probability distribution 52 for the subcohort for the patient. The vertical axis designates probabilities (here: of contracting coronary heart disease) and the horizontal axis designates the body mass index, BMI.

The estimator function f_{θ} can also be any type of learned function which is, for example, derived from a machine learning method. Hence, the estimator function f_{θ} can be based on linear or logistic regression, machine learning, support vector machines and/or the like. The estimator function f_{θ} can be trained on the total population or on the subcohort. The estimator function f_{θ} can be trained to output a single value (instead of at least two values) or to output a plurality of values, as described in the foregoing.

In the step S50, if the estimator function f_{θ} outputs a probability distribution 51, 52, symbolized by yₑₛₜ as illustrated on the left-hand side of FIG. 3, a probability distribution 53, 54 as illustrated on the right-hand side of FIG. 3 can be calculated for the task function g, i.e. g(x, yₑₛₜ). In other words, in the present example, the probability distributions 53, 54 indicate the risk of the patient suffering from coronary heart disease in the next five years.

The probability distribution 53 is based on the probability distribution 51 for the general population and the probability distribution 54 is based on the probability distribution 52 for the subcohort.

FIG. 3 illustrates how, for example, a shift in the probability distribution 51, 52 on the horizontal axis between the values for the general population and the subcohort substantially results in a narrowing and tapering of the probability distribution 54 for the subcohort compared to the probability distribution 53 for the general population. In the example shown, the plurality of values g1, g2, etc., for the task function, i.e. the probability distribution 54, ranges between 10.5% and 21.6%.

In a step S60, a relevance metric is calculated for the at least one of the at least one insufficient data field and/or the value thereof for the medical task. For example, the entire width or the full width at half maximum (FWHM) or the distance of the zeros from a center of the probability distribution 54 and/or the like can be calculated as a relevance metric.

In a step S70, the relevance metric is compared with a relevance threshold value, or, in other words, it is determined whether or not the specific relevance metric is greater than or equal to a relevance threshold value (or, in other variants, greater than the relevance threshold value).

In a step S80, an output signal 71 based on the at least two calculated results is output if it was determined that the relevance metric is greater than or equal to the relevance threshold value. As described in the foregoing, a (different) output signal 71 can also be output if it was determined that this is not the case.

For example, in the present case, the relevance threshold value can be a threshold value of 1% for the spacing between the zeros of the probability distribution 54. Since, in the example in FIG. 3, this distance is 21.6%−10.5%=11.1% and 11.1%>1%, in this case, it is determined that the relevance metric is greater than the relevance threshold value.

In this example, the relevance threshold value is selected as very low in order to filter out only trivial small variations, which would only confuse the physician.

The output signal 71 can include or consist of any number of signals, such as, for example, warning signals, information signals, control signals etc.

For example, the output signal 71 can control a display, which displays probability distributions 52, 54 in FIG. 3 or even all probability distributions 51, 52, 53, 54 in FIG. 3 to a physician, so that the physician is able to determine whether or not the change in the values for the insufficient data field and/or the change in the results for the task function g is acceptable.

Additionally or alternatively, a mean value and a standard deviation of the risk can be calculated based on the probability distribution 51, 52 for the results of the task function g, for example using a Monte Carlo simulation. This result, for example an average risk of 18.3%±2.7%, can also be depicted as the result of the control of the display by the output signal 71. As depicted in FIG. 3, in each case a comparison between the probability distributions 51, 53 for the general population and the probability distributions 52, 54 for the subcohort can be displayed for both the result of the estimator function f_{θ} and/or for the result of the task function g and can optionally also be automatically analyzed.

The output signal 71 can also contain or consist of a warning signal or a control signal, as was explained in the foregoing. For example, the output signal 71 can include (or consist of) a warning signal, which displays to a physician that the result of the task function g is evaluated as too uncertain. The output signal 71 can also pause the performance of the method according to the first aspect of the present invention. In some variants, the output signal 71 can automatically perform a process for obtaining improved values (or any value, in the case of missing values) for the insufficient data field.

In some variants, a plurality of relevance threshold values can be provided and a different output signal 71 (or different output signals) can be output depending upon how the calculated relevance metric is arranged in comparison to each of the relevance threshold values.

For example, if the relevance metric exceeds a first relevance threshold, the output signal 71 can be output such that one or both of the probability distributions 53, 54 in FIG. 3 are shown to a physician, i.e. probability distributions for the result of the task function g in accordance with the medical task to be performed.

If the relevance metric exceeds an optional second threshold value, which is greater than the first relevance threshold value, the output signal 71 can be output such that a warning is displayed to the physician indicating that the method should not be continued due to excessive uncertainties and/or such that the method is automatically paused.

If the relevance metric an optional third relevance threshold value (which can be located anywhere in relation to first relevance threshold value and the optional second relevance threshold value and which can also be the same as one or both of these relevance threshold values), the output signal 71 can be generated such that it includes a request signal, which initiates a workflow for obtaining improved values for the insufficient data field.

Preferably, at least the first and third relevance threshold value are set and the third relevance threshold value is lower than the first relevance threshold value. This means that, for relevance threshold values between the first relevance threshold value and the third relevance threshold value, the physician does not need to bother with probability distributions but can be provided with a single value (for example based on a median, an average or a specific quantile or percentile for the insufficient data field). However, in this example, because the first relevance threshold value is exceeded, it is still possible for measures to be taken in order to obtain an improved value. This is based on the consideration that, although this may be of little relevance for the specific present task, it is generally desirable to obtain the best possible values for all the data fields.

Different tasks can be provided with different relevance threshold values in one and the same implementation of the method or of the computer system 100. For example, relevance threshold values can be set higher for medical tasks that are represented by task functions which are known to (or even have to) generate results with high degrees of uncertainty since only a rough estimate is expected from these medical tasks anyway.

With reference to the computer system 100 in FIG. 2, the input interface 110 can be configured to perform the steps S10 and S20 as described in the foregoing and the computing apparatus 150 can be configured to perform the steps S30 to S70. If the specific relevance metric is greater than or equal to the threshold value, the computing apparatus 150 can control the output interface 190 to output 180 an output signal 71 based on the at least two results calculated for the medical task to be performed. The output signal 71 can be generated by the computing apparatus 150 and/or the output interface 190.

The computing apparatus 150 can be embodied as any device or any means for calculating data, in particular for executing software, an app or an algorithm. For example, the computing apparatus 150 can have at least one processor unit, such as, for example, at least one central processing unit (CPU) and/or at least one graphics processing unit (GPU) and/or at least one field-programmable gate array, FPGA and/or at least one application-specific integrated circuit, ASIC, and/or include or consist of any combination of the above.

The computing apparatus 150 can also have a random-access memory that is operatively coupled to the at least one processing unit is and/or can have a non-volatile storage medium, which is operatively coupled to the at least one processing unit and/or the random-access memory. The computing apparatus 150 can be implemented as a local device, a remote device (such as, for example, a server, which is remotely connected to a local client or terminal with a user interface) or can be embodied as a combination thereof. Part of the computing apparatus 150 or the entire computing apparatus 150 can also be implemented by a cloud computing system. The input interface 110 and/or the output interface 190 can also be integrated in the computing apparatus 150.

The computer system 100 can also have at least one output device, for example a display, a loudspeaker, headphones or the like. The output signal 71 can control the output device to output information to a user (usually a physician) based on the at least two results calculated for the medical task, preferably a display device (such as, for example, a computer screen, a touchscreen or the like) for depicting the information graphically.

For each of the method steps S30 to S70, a corresponding software module can be provided, which is stored in the computing apparatus 150 and executed by the computing apparatus 150, for example an insufficiency calculation module for determining whether an insufficient data field is present, a relevance metric determination module, an estimator function calculation module, a relevance metric comparison module and/or a output interface control module. Some or all of these modules of the computing apparatus 150 can be implemented by a cloud computing system.

FIG. 4 shows a schematic block diagram of a computer program product 200 according to the third embodiment of the present invention, i.e. a computer program product 200, which includes a executable program code 250, which is embodied, when executed, when it is executed by computing apparatus 150 to perform the method according to FIG. 1.

FIG. 5 illustrates a non-volatile, computer-readable data storage medium 300 according to the fourth embodiment of the present invention, i.e. a data storage medium 300, which includes executable program code 350, which is embodied, when it is executed by a computing apparatus 150, to execute the method according to FIG. 1.

In the preceding detailed description, various features have been combined in order to keep the description brief. It should be understood that the foregoing description is intended to be illustrative and not restrictive. It is intended to include all alternatives, modifications and equivalents. Those skilled in the art will implicitly read many other examples when considering the foregoing description and consider the various variants, modifications and options as described in the foregoing.

Although the invention has been illustrated in greater detail using the example embodiments, the invention is not limited by the disclosed examples, and a person skilled in the art can derive other variations therefrom without departing from the scope of protection of the invention.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A computer-implemented method for performing or supporting a medical task, comprising:
obtaining a medical task to be performed;
obtaining a plurality of values for a plurality of data fields of a number of available data fields related to medical data;
determining whether, after the obtaining of the plurality of values for the plurality of data fields, at least one insufficient data field is present among the plurality of data fields, wherein an insufficient data field is a data field for which no value was obtained or for which a value obtained is insufficient according to at least one quality criterion;
determining, upon determining that at least one insufficient data field is present among the plurality of data fields, a relevance metric for the medical task to be performed, for at least one of the at least one insufficient data field and a value of the at least one insufficient data field;
providing, via an estimator function, at least two different values for the at least one of the at least one insufficient data field;
calculating at least two results for the medical task to be performed, the at least two results being based on the at least two different values provided;
determining whether the relevance metric determined reaches or exceeds a relevance threshold value; and
outputting, upon the determining indicating that the relevance metric reaches or exceeds the relevance threshold value, an output signal based on the at least two results calculated.

2. The method of claim 1, wherein the calculating of the at least two results of the medical task to be performed includes a calculation of a result for each of the at least two different values for the at least one of the at least one insufficient data field.

3. The method of claim 1, wherein the at least two values provided for the at least one of the at least one insufficient data field include a minimum value and a maximum value.

4. The method of claim 1, wherein the at least two different values provided for the at least one of the at least one insufficient data field include at least two different quantiles.

5. The method of claim 1, wherein at least one of the at least two results for the medical task to be performed and the at least two different values provided by the estimator function, are based on a general population or on a subcohort for a patient for whom the medical task is to be performed.

6. The method of claim 1, wherein the at least one insufficient data field is a data field including binary values or include values including a linear effect on the medical task to be performed.

7. The method of claim 1, wherein the calculating of the at least two results for the medical task to be performed is performed based on a probability distribution provided by the estimator function for the at least one of the at least one insufficient data field.

8. The method of claim 1, wherein, upon at least one of the at least two results calculated or a size derived from at least one of the at least two results calculated, meets a set condition, at least one of a warning signal and a control signal is automatically output, at least one of indicating necessity for an improved value for the at least one insufficient data field to be obtained and performing a control function to obtain an improved value.

9. The method of claim 1, wherein one of the at least one quality criterion includes whether a value was generated by at least one of optical character recognition, OCR, and by natural language processing.

10. The method of claim 9, wherein one of the at least one quality criterion includes whether reliability information for the at least one of optical character recognition, OCR, and the natural language processing, is above a threshold value.

11. A computer system for performing or supporting a medical task, comprising:
an output interface;
an input interface configured:
to obtain a medical task to be performed; and
to obtain a plurality of values from a plurality of data fields of a number of available data fields, related to medical data;
a computing apparatus configured:
to determine, after the obtaining of the plurality of values of the plurality of data fields, at least one insufficient data field is present among the plurality of data fields, wherein an insufficient data field is a data field for which no value was obtained or for which a value was obtained which is insufficient according to at least one quality criterion,
to determine, upon determining that at least one insufficient data field is present among the plurality of data fields, a relevance metric for at least one of the at least one insufficient data field and the value of the at least one insufficient data field, for the medical task;
to use an estimator function to provide at least two different values for the at least one of the at least one insufficient data field;
to calculate at least two results for the medical task to be performed based upon the at least two different values provided for the at least one of the at least one insufficient data field;
to determine whether the relevance metric determined is greater than or equal to a relevance threshold value, and
to control, upon the determining indicating that the relevance metric reaches or exceeds the relevance threshold value, the output interface to output an output signal based on the at least two results calculated.

12. A non-transitory computer program product storing executable program code, which when executed by at least one processor, configures the at least one processor to perform the method of claim 1.

13. A non-volatile, computer-readable data storage medium storing executable program code, which when executed by at least one processor, configures the at least one processor to perform the method of claim 1.

14. The method of claim 2, wherein the at least two values provided for the at least one of the at least one insufficient data field include a minimum value and a maximum value.

15. The method of claim 2, wherein the at least two different values provided for the at least one of the at least one insufficient data field include at least two different quantiles.

16. The method of claim 2, wherein at least one of the at least two results for the medical task to be performed and the at least two different values provided by the estimator function, are based on a general population or on a subcohort for a patient for whom the medical task is to be performed.

17. The method of claim 2, wherein the at least one insufficient data field is a data field including binary values or include values including a linear effect on the medical task to be performed.

18. The method of claim 2, wherein the calculating of the at least two results for the medical task to be performed is performed based on a probability distribution provided by the estimator function for the at least one of the at least one insufficient data field.

19. The method of claim 2, wherein one of the at least one quality criterion includes whether a value was generated by at least one of optical character recognition, OCR, and by natural language processing.

20. The method of claim 19, wherein one of the at least one quality criterion includes whether reliability information for the at least one of optical character recognition, OCR, and the natural language processing, is above a threshold value.
